(19) United States
(12) Patent Application Publication
(10) Pub. No.: US 2025/0120902 A1
(43) Pub. Date: Apr. 17, 2025

(54) POSTBIOTIC FERMENT COMPOSITION FOR ROOT, SCALP, AND HAIR CARE AND METHOD OF PREPARING AND USING THE

(71) Applicant: THE ROOTIST INC., Palm Beach Gardens, FL (US)

(72) Inventors: Alison R. Cutlan, Brooklyn, FL (US); Kseniya Popova, Mahwah, NJ (US)

(73) Assignee: THE ROOTIST INC., Palm Beach Gardens, FL (US)

(21) Appl. No.: 18/918,007

(22) Filed: Oct. 16, 2024

Related U.S. Application Data
(60) Provisional application No. 63/544,444, filed on Oct. 16, 2023.

(51) Int. Cl. A61K 8/9789; A61K 8/9728; A61Q 5/00

(52) U.S. Cl. CPC A61K 8/9789; A61K 8/9728; A61Q 5/00; A61K 2800/85

(57) ABSTRACT

Provided is a postbiotic ferment composition for treatment of root, scalp and hair care using fermentation bacteria of Lactobacillus species, a method of manufacturing the same, and a scalp and hair treatment method using the same. A method of preparing a postbiotic ferment composition for treatment of root, scalp and/or hair may include: obtaining an extract from a plurality of raw materials, wherein the plurality of raw materials comprises Panax ginseng root, Ganoderma lucidum, Centella asiatica, Humulus lupulus, coffee arabica seed, and Scutellaria baicalensis root; combining the obtained extract with Pisum sativum and Oryza sativa to obtain a combined product; and inoculating the combined product with one or more bacterial species of Lactobacillus to induce fermentation and obtain the postbiotic ferment composition for treatment of root, scalp, and/or hair.

### CROSS-REFERENCE TO RELATED APPLICATION(S)

The present application claims priority to and/or benefit of U.S. provisional patent application No. 63/544,444 filed on Oct. 16, 2023, the entire contents of which is hereby incorporated by reference.

### TECHNICAL FIELD

The present disclosure relates to a postbiotic ferment composition for treatment of root, scalp, and hair care using fermentation via Lactobacillus species bacteria, a method of preparing the same, and a scalp and hair treatment method using the same.

### BACKGROUND

A large portion of the general population has or will experience in their lifetime a number of issues related to their scalp and/or hair. Some of the main concerns include sensitivities, inflammation and itchiness caused by scalp dryness, irritations from hair products, dandruff or underlying skin conditions like eczema or psoriasis. Excessive oil production can lead to greasy hair and may contribute to issues like seborrheic dermatitis and scalp acne. Thinning hair and hair loss is also widely experienced due to several factors such as stress, hormonal changes, genetics while hair dullness, breakage and split ends occur from over styling, chemical treatments and lack of proper care.

However, haircare products commonly found on the market (i.e., shampoos, conditioners, serums, and styling products, etc.) often contain ingredients not especially suited for the biological systems of the scalp and hair, and can lead to irritations, microbiome imbalances and barrier disruptions that cascade into other compounding problems. It is imperative to create a novel product and method for treatment of haircare by taking a biological approach—one that is safer, holistic, and far more effective.

Fermentation is used for food and beverage production and for nutritional and wellness purposes. Fermentation is a natural process whereby beneficial microbes digest and transform substances into smaller, more bio-available forms, increasing their penetration and potency when consumed. Moreover, fermenting microbes produce secondary metabolites such as polysaccharide sugars, amino acids, enzymes, and vitamins that enrich the total nutritional value of the surrounding media. The resulting output of fermentation is referred to as a ‘postbiotic,’ and health benefits of postbiotics (and fermented foods in general) can be attributed, in part, to their positive influence on resident microbial communities among many other benefits.

Consequently, fermentation has emerged as a powerful method for developing new, sustainable, and biologically-oriented active ingredients and postbiotics with improved safety, compatibility, and efficacy for our bodies.

### SUMMARY OF DISCLOSURE

The present disclosure provides a fermented postbiotic having preventive and treatment effect in addressing various root, scalp, and hair wellness concerns. The fermented postbiotic and resulting compositions made therefrom help foster a balanced and healthy scalp microbiome—promoting the growth of beneficial microorganisms while suppressing harmful ones. This proves particularly effective in combating issues such as scalp inflammation including redness, itchiness, and scalp sensitivities. Moreover, it has been discovered that postbiotics can play a vital role in strengthening the skin's barrier function and aiding moisture retention within the scalp, and its dense nutrient content would nourish the root, scalp, and hair, thereby promoting healthier hair growth while enhancing strength and shine of hair.

Utilizing Lactobacilli (probiotic) fermentation, a novel nutritive and active botanical postbiotic product can be produced that can holistically support the biological functioning of the root, scalp and hair and serve as a base for biologically-minded haircare products.

This type of biotechnological processing often involves targeted fermentation whereby specific probiotic bacterial strains are combined with (or without) a substrate to enhance skin health benefits. The present disclosure describes a novel fermentation product combining eight different plant or fungal ingredients and using one or more strains of Lactobacilli bacteria species to enhance the health and wellness of the root, scalp and hair.

The effects of the present disclosure are not limited to the above effects but should be construed to include all effects that can be inferred from the configuration of the invention disclosed in the description or claims of the present disclosure.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flowchart illustrating a process of obtaining an extract from raw materials according to an embodiment of the present disclosure.

FIG. 2 is a flowchart illustrating a process of obtaining a postbiotic ferment composition from the raw material extract according to an embodiment of the present disclosure.

FIG. 3 illustrates a graph showing experimental results regarding hair follicle organoid diameter.

FIG. 4 illustrates a graph showing experimental results regarding hair follicle organoid growth rate.

FIGS. 5-7 are show images of experimental results regarding hair follicle organoid diameter and growth rate.

### DETAILED EXPLANATION OF DISCLOSURE

The method for manufacturing a scalp and hair treatment composition is characterized by including: obtaining an extract from a plurality of different plant or fungal raw materials, combining the extract with one or more proteins, which may be plant proteins, inoculating one or more species of Lactobacilli bacteria into the combination of the extract and plant proteins to induce fermentation. The bacteria may be one or more species of Lactobacillus, such as Lactobacillus plantarum and/or Lactobacillus casei.

The plurality of raw materials and proteins may include: Panax ginseng, Ganoderma lucidum, coffee arabica, Scutellaria baicalensis, Centella asiatica, Humulus lupulus, Pisum sativum, and Oryza sativa. A brief description of each ingredient is described herebelow.

1. Red Ginseng Root (Panax ginseng)

Panax ginseng (also called Korean or Red Ginseng) is an adaptogenic root having energizing, revitalizing and anti-aging effects for roots and scalp. It promotes collagen and hyaluronic acid synthesis, boosts circulation, stimulates hair growth, combats hair loss, and maintains a healthy root, scalp, and hair. Rich in antioxidant and anti-inflammatory compounds, Panax ginseng may reduce circulating cortisol, a known factor in stress-induced hair loss.

Red Ginseng promotes hair growth by preventing apoptosis of hair follicle cells and inhibiting 5-α reductase, and it also increases hair follicle density. Key bioactive compounds in Panax ginseng are ginsenosides and cysteine.

2. Reishi Mushroom (Ganoderma lucidum)

Reishi mushroom (Ganoderma lucidum) has adaptogenic properties and promotes overall well-being, reducing inflammation, and supporting the immune system. In the context of root, scalp, and hair health, reishi mushroom may help to nourish the hair follicles and support a healthy scalp environment. Reishi mushroom extracts are rich in peptides, amino acids, and polysaccharides, such as beta glucans, that repair and restore the moisture barrier, along with ganoderic acid, a triterpenoid that inhibits 5a-reductase activity, which blocks the conversion of DHT (dihydrotestosterone) from testosterone, reducing hair follicle loss.

3. Coffee Seed (Coffee arabica)

Coffee beans are rich in antioxidants and caffeine, which can have stimulating effects. Caffeine promotes hair growth by extending the anagen (growth) phase of the hair follicles and stimulating blood circulation in the scalp. Additionally, the antioxidant properties of coffee help protect the scalp and hair follicles from oxidative stress.

4. Skullcap Root (Scutellaria baicalensis)

Skullcap root (Scutellaria baicalensis) is rich in flavonoids and has anti-inflammatory and antioxidant properties. Skullcap root provides calming and soothing effects on the scalp and promotes a healthier scalp environment in addition to increasing hair follicle development at the roots. Fermentation of Skullcap may convert key flavonoids (such as Baicalin and Wogonoside) to their more powerful and active forms, increasing their bioactivity on the body.

5. Gotu Kola (Centella asiatica)

Centella asiatica has wound healing and skin rejuvenating properties. It supports scalp health by reducing inflammation, improving circulation, and promoting collagen synthesis, which can promote scalp and root health and therefore healthier hair growth.

6. Hops Flower (Humulus lupulus)

Hops (Humulus lupulus) possess antioxidant, anti-inflammatory and anti-microbial and strengthening properties. In the context of haircare, its rich source of silica improves hair strength and shine while its bitter acids, polyphenols and flavonoids contribute to maintaining a balanced and calm scalp environment with possible dandruff control.

7. Pea Protein (Pisum sativum)

Pea protein contains high levels of amino acids including glutamic acid, aspartic acid, arginine and lysine, all of which have vital properties for hair; including repair and improving strength, elasticity, nourishment and hydration to the hair follicles and shaft. Hydrolyzed pea protein allows for tinier particles of proteins to bond to the hair, linking together to form a protective structure that prevents cuticle cracking and damage and boosts the hydration and moisture retention in each strand.

8. Rice Protein (Oryza sativa)

Rice is rich in amino acids cysteine and methionine (sulfur rich amino acids) and provides nourishment to the hair shaft, helping strengthen and repair damaged hair. Hydrolyzed rice peptides increased the brightness of hair. Fermentation simplifies the rice proteins, amplifying amino acids and short chain FA to penetrate, nourish and repair hair.

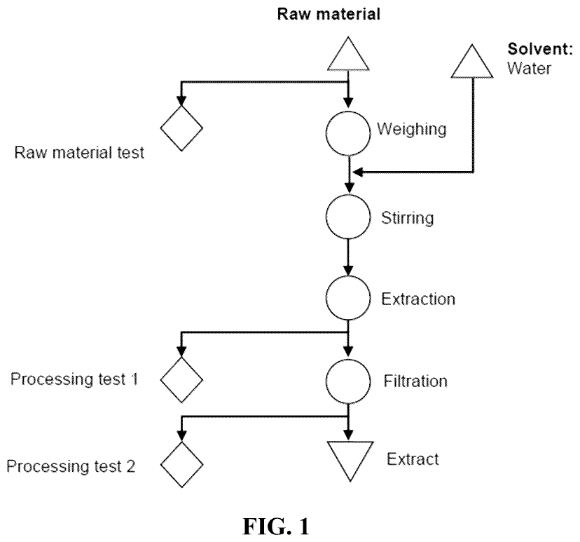

FIG. 1 schematic flowchart of a process of extraction according to an embodiment of the present disclosure. The process shown in FIG. 1 shows a schematic representation, but the present disclosure is not limited thereto. The term “extract” as used herein refers to a material extracted from a raw material by any method, and includes, without limitation, an extract solution obtained thereby, a concentrate obtainable therefrom, and a dried or powdered form of the concentrate.

The extract may be obtained by extraction from a raw material or a dried form thereof. The raw material of the extract may include, without limitation, cultivated or commercially available ones.

In extracting the raw materials to obtain the extract, the extraction method used in the present disclosure may include any known conventional extraction method, such as solvent extraction, ultrasonic extraction, high-pressure extraction, ultra-high pressure extraction, filtration extraction, or reflux extraction. Preferably, the extract may be prepared by using solvent extraction, infusion, or reflux extraction. The extraction process may be repeated several times, and thereafter, an additional step of concentration or freeze-drying may be conducted. Specifically, the obtained extract may be concentrated under reduced pressure to form a concentrate, and the concentrate may be freeze-dried and powdered with a grinder into a high-concentration extract powder. The extract of the present disclosure includes a fraction obtained by additional fractionation of the extract or a fermented product obtained by an additional process such as fermentation of the extract.

The extract may be obtained from the plant or fungal raw materials described hereabove, such as Panax ginseng, Ganoderma lucidum, coffee arabica, Scutellaria baicalensis, Centella asiatica, Humulus lupulus. A plurality of factors were considered in selecting the raw materials to be included in the postbiotic ferment composition of the present disclosure, including amount of sebum in the scalp, microbiome diversity and balance, and barrier health. Further, consideration was given to improve scalp/hair health at all four main phases of the hair cycle (anagen, catagen, telogen, and exogen), with respect to hormones, genetics, environment, and stress, which are known causes of hair thinning and/or loss.

After the extract is obtained, the extract may be combined with one or more proteins, preferably plant proteins. In one embodiment, Pisum sativum and Oryza sativa are combined with the extract to obtain a combined product.

Thereafter, the combined product is inoculated with one or more Lactobacillus bacteria species to induce fermentation. The Lactobacillus bacteria species may be Lactobacillus plantarum or Lactobacillus casei, and more preferably, the Lactobacillus bacteria species for inducing fermentation may be both Lactobacillus plantarum and Lactobacillus casei.

In more detail, the raw materials are combined in following exemplary weight ratios for the extraction, making a total of 10% to 25% w/w: Centella asiatica (Gotu Kola) 2 to 4%, Humulus lupulus (Hops) 2 to 4%, Scutellaria baicalensis (Skullcap) Root 2 to 5%, Panax ginseng Root 1%-5%, Ganoderma lucidum (Reishi Mushroom) 1 to 4%, Coffea arabica (Coffee) Seed 0.25-2%, with the remainder as an extraction solvent, such as water.

The raw materials may be combined with 75% to 90% solvent (water), heated and may be taken through a two-part extraction process starting with Ceramic Extraction that uses consistent gentle heat, followed by Megasonic Extraction that relies on high frequency sound waves (e.g., 700 kHz and 1.2 MHz) to gently yet effectively extract phytochemicals via cavitation. The above extraction methods are indicated as examples only and other extraction methods known in the art may be used.

After the extraction is complete, the extraction mixture is filtered, separating the spent plant material from the extraction liquid. To this filtrate, Pisum sativum (pea) seed protein and Oryza sativa (rice) protein powders were incorporated at 0.25% to 1% w/w each, for a total of 0.5% to 2% w/w to obtain a final liquid extract.

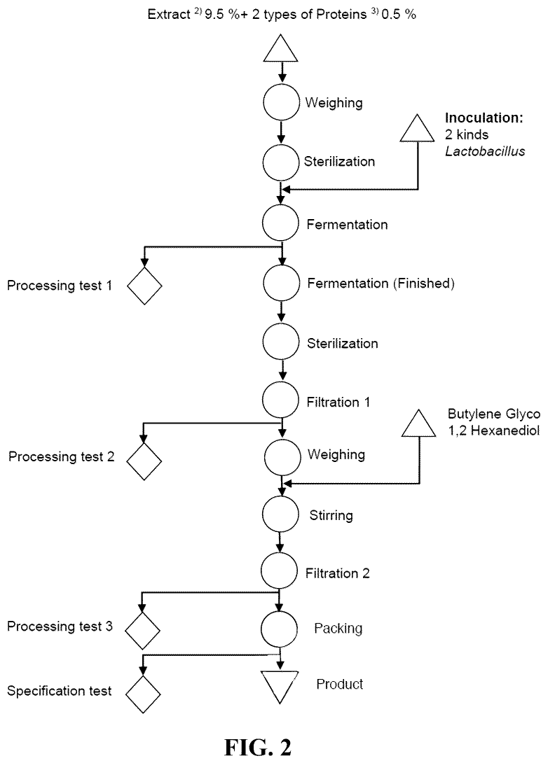

The liquid extract filtrate described above is then sterilized and then inoculated with one or more bacteria species of genus Lactobacillus. The Lactobacillus bacteria species may be Lactobacillus plantarum or Lactobacillus casei. In one embodiment, both Lactobacillus plantarum and Lactobacillus casei may be used to ferment the liquid extract. For example, of 0.5% to 1% w/w Lactobacillus plantarum and 0.5% to 1% w/w Lactobacillus casei may be used to induce a fermentation process and obtain a postbiotic fermentation product. A schematic flowchart of the fermentation process according to an embodiment of the present disclosure is shown in FIG. 2.

Lactobacillus plantarum is a unique species of Lactobacillus that is commonly found on plants and vegetation (and subsequently is a part of the human gut microbiome), e.g., from apple. Live bacteria and the postbiotic lysates from Lactobacillus plantarum promote peacekeeping bacteria on the skin microbiome and promote healthy functioning of the skin, such as improved barrier functioning and hydration, skin immunity, reducing acne, and providing soothing effects.

Lactobacillus casei is a unique strain of Lactobacillus which can be derived from vegetable kimchi made of cabbage, and boosts skin barrier functioning and health and has a balancing effect on the microbiome.

Fermentation may be carried out for 24 to 72 hours and may reach a pH in the range of 3.5 to 5.

Once fermentation is complete, the broth is heat sterilized to lyse the bacterial cells and taken through a filtration process to obtain the postbiotic ferment composition.

A glycol and a preservative may be then added to this postbiotic filtrate, re-filtered through a sterile filter (0.3 micron), and packaged.

The postbiotic fermentation product obtained after fermentation of the extracts may be provided in the form of a liquid broth or in powder form.

### Experimental Example 1 (Pre-Fermentation Extract)

To measure bioactive activity of the postbiotic ferment composition of the present disclosure and the effect of fermentation on the raw materials/extracts, Experimental Example 1, which is the pre-fermentation extract, is obtained from the following six raw materials at the indicated wt %:
2.5 wt % Centella asiatica;
2.5 wt % Humulus lupulus;
2.5 wt % Scutellaria baicalensis root;
1 wt % Panax ginseng root;
1 wt % Ganoderma lucidum; and
0.5 wt % coffee arabica seed, with water as the remainder as the extraction solvent.

The above mixture is heated and taken through a two-part extraction process starting with Ceramic Extraction that uses consistent gentle heat, followed by Megasonic Extraction that relies on high frequency sound waves (e.g., 700 kHz and 1.2 MHz) to gently yet effectively extract phytochemicals via cavitation.

After the extraction is complete, the extraction mixture is filtered, separating the spent plant material from the extraction liquid. To this filtrate, Pisum sativum and Oryza sativa protein powders were incorporated at 0.25% w/w each, for a total of 0.5% w/w to obtain a final liquid extract, i.e., Experimental Example 1.

### Experimental Example 2 (Post-Fermentation—Postbiotic Ferment Composition)

The final liquid extract of Experimental Example 1 is inoculated with 0.5% w/w Lactobacillus plantarum and 0.5% w/w Lactobacillus casei to induce fermentation. The fermentation is carried out for 24 hours and processed for additional 48 hours, the resulting product is then sterilized and filtrated to obtain the postbiotic ferment composition, i.e., Experimental Example 2.

### Experimental Procedures

### In Vitro Experimental Data

Samples Experimental Example 1 and Experimental Example 2 were obtained for chemical and in vitro analysis.

The bioactivity of the postbiotic ferment was evaluated through a number of tests including: 1) total polyphenol, flavonoid, beta glucan and ginsenoside content, 2) in vitro cellular assays for anti-oxidation and anti-inflammatory action, and 3) in vitro cell PCR studies investigating skin moisturization, firming and inflammation associated with acne. The results are as follows:

### Chemical Analysis:

1) Total polyphenol, flavonoid, beta glucan and ginsenosides were measured for Experimental Example 1 and Experimental Example 2 samples, and in all instances, fermentation increased the concentration of these compounds with beta glucan content showing the greatest enhancement.

TABLE 1

| Content Analysis | B-Glucan | Polyphenol | Flavonoid | Ginsenosides |
|---|---|---|---|---|
| Experimental | 112.31 | 70.27 | 55.03 | 16.60 |
| Example 1 | mg/kg | mg/kg | mg/kg | mg/kg |
| Experimental | 295.32 | 93.00 | 66.28 | 19.30 |
| Example 2 | mg/kg | mg/kg | mg/kg | mg/kg |

The postbiotic ferment composition of the present disclosure includes 10 different identified forms of ginsenosides present, the highest being Ginsenoside Rb1, which is known to prevent hair loss and to stimulate the proliferation of hair follicles.

2) In vitro cellular assays of Experimental Example 1 and Experimental Example 2 samples were run for antioxidant activity (Intracellular ROS Inhibition Assay) and anti-inflammatory activity (No Synthesis Inhibition Assay). The tests showed the filtrates showing marked activity, with Experimental Example 2 samples showing higher activity, i.e., enhancement through fermentation.

The antioxidant (ROS Inhibition Assay) detail: Reactive oxygen species (ROS) are produced by mitochondrial respiration when intracellular energy is produced; when excessive, they cause cellular damage by changing the intracellular environment. The test method uses a fluorescence measurement method employing DCFH-DA (2,7-dichlorofluorescein-diacetate). DCFH-DA easily penetrates cells as a non-polar molecule and is separated into DCFH, a non-fluorescent polar molecule, by esterase. Thereafter, when the cells (HDFn) are oxidized by active oxygen, they are converted to fluorescent DCF; using a fluorometer, the antioxidant efficacy can be evaluated.

The anti-inflammatory (NO Synthesis Inhibition Assay) detail: NO synthesis inhibition assay: Inflammation is an immune response to protect the body from damage caused by external stimuli. It is a critical system to alleviate damage to subcutaneous tissue and maintain the body's homeostasis. However, when this inflammatory reaction is excessively activated, it contributes to developing various skin diseases. RAW 264.7 macrophage cells are treated with LPS (lipopolysaccharide), which is known as an inflammatory substance. The degree of inhibition of the sample's inflammatory reaction can be measured using the Griess reagent reaction method for NO produced by iNOS.

### Results of In Vitro Cellular Assays:

TABLE 2

| In vitro assay | Antioxidant | Anti-inflammatory |
|---|---|---|
| Experimental Example 1 | 51.0% (Conc. 50%) | 83.8% (Conc. 50%) |
| Experimental Example 2 | 62.5% (Conc. 50%) | 91.1% (Conc. 50%) |

As a result, the Experimental Example 1 showed an anti-oxidant efficacy of 51.0% with a 50% (v/v) concentration. The Experimental Example 2 showed an antioxidant efficacy of 62.5% with a 50% (v/v) concentration.

As a result, the Experimental Example 1 showed an anti-inflammatory efficacy of 83.8% with a 50% (v/v) concentration, and the Experimental Example 2 showed an anti-inflammatory efficacy of 91.1% with a 50% (v/v) concentration.

These results indicate that the postbiotic ferment has marked activity against oxidative stress and inflammation and that this activity was enhanced through the fermentation process. Antioxidant and Anti-inflammatory activities are important for overall health and wellness of the structures of the roots, scalp, and hair.

3) In vitro cellular PCR analysis using the RT-qPCR method was used to evaluate gene expression related to skin moisturization and skin firming on the [Experimental Example 1 and Experimental Example 2 samples.

Quantitative real-time polymerase chain reaction (RT-qPCR) is a method that repeatedly synthesizes and amplifies specific DNA segments of gene sequences, facilitating the measurement of gene expression in a cell.

### 3A. Moisturization Genes Evaluated Included Hyaluronic Acid Synthase (HAS-2) and Aquaporin-3 (AQP-3):

Hyaluronic acid synthase-2 (HAS-2): Hyaluronic acid synthase-2 (HAS-2) is responsible for the synthesis of hyaluronic acid (HA), which is a type of glycosaminoglycan (GAG). HA, along with collagen and elastin, plays a vital role in maintaining skin/scalp moisture and healthy functioning as one of the three main components of the dermis. The hyaluronic acid synthase (HAS) is responsible for the synthesis of HA, and HAS-2 specifically plays a crucial role in this process.

Aquaporin-3 (AQP-3): AQP is an integral membrane protein that channels the transfer of water across the membrane. In the epidermal layer of the skin and scalp, AQP-3 is primarily responsible for regulating the movement of water and glycerol between cells. As the skin ages, the expression of AQP-3 decreases, which causes dry skin/scalp.

TABLE 3
3A Results

| | Moisturization | |
|---|---|---|
| In vitro PCR assay | HAS-2 | AQP-3 |
| Experimental Example 1 | 149.7% (12.5% conc.) | 218.2% (12.5% conc.) |
| Experimental Example 2 | 238.7% (12.5% conc.) | 667.4% (12.5% conc.) |

At 12.5% (v/v), of the Experimental Example 1, the expression of the HAS-2 gene increased by 149.7% over the control, and at 12.5% (v/v), of the Experimental Example 2, the expression of the HAS-2 gene increased by 238.7% over the control.

This result indicates that the combination of the eight raw ingredients increases the synthesis of the glycosaminoglycan and Hyaluronic acid within the dermis, and the resulting postbiotic ferment enhanced this activity even more significantly. Hyaluronic acid plays a vital role in maintaining the moisture and healthy functioning within the root and scalp.

At 12.5% (v/v) of the Experimental Example 1, the expression of the AQP-3 gene increased by 218.2% over the control, and at 12.5% (v/v) of the Experimental Example 2, the expression of the AQP-3 gene increased by 667.4% over the control.

The significant increase in the expression of the AQP-3 gene in the Experimental Example 1 and Experimental Example 2 samples indicates that the postbiotic ferment composition has the ability to enhance intracellular hydration in the skin and scalp through the aquaporin expression. Proper hydration helps to prevent aging, irritation and damage and is essential for healthy looking skin/scalp.

### 3B. Skin Firming Genes Evaluated Include Decorin [DCN] and Lumican [LUM]:

Decorin (DCN): Decorin strengthens the cross-linking of collagen fibers, preventing collagen degradation by MMP-1. This supports structural integrity of the underlying structures of the skin, promoting healthy root anchoring and scalp health by increasing the secretion of growth factors involved in cellular regeneration.

Lumican (LUM): Lumican is a protein that contributes to stabilizing collagen fibers and aiding in the restoration of the dermis and extracellular matrix (ECM), providing root anchoring support and skin/scalp elasticity. Reduced expression of lumican can impair the formation of type 1 collagen, resulting in skin aging.

TABLE 4
3B Results

| | Skin Firming | |
|---|---|---|
| In vitro PCR assay | DCN | LUM |
| Experimental Example 1 | 71.7% (12.5% conc.) | 70.5% (12.5% conc.) |
| Experimental Example 2 | 144.5% (12.5% conc.) | 120.4% (12.5% conc.) |

At 12.5% (v/v) of the Experimental Example 1, the expression of the DCN gene increased by 71.7% over the control, and at 12.5% (v/v) of the Experimental Example 2, the expression of the DCN gene increased by 144.5% over the control.

At 12.5% (v/v) of the Experimental Example 1, the expression of the LUM gene increased by 70.5% over the control, and at 12.5% (v/v) of the Experimental Example 2, the expression of the LUM gene increased by 120.4% over the control.

From these in vitro experimental results we can see that the unique herbal filtrate including eight specific natural ingredients increases DCN and LUM gene expression, and this activity is increased even more significantly upon fermentation. This indicated that the unique postbiotic ferment will help enhance and restore the underlying structures of the skin/scalp, including strengthening root (follicle) anchoring and support resulting in greater hair retention, growth, and health of hair.

### 4. Amino Acid Analysis

Amino acid analysis of the postbiotic ferment was conducted and showed a high level of Arginine (30.5 mg/kg). Arginine has powerful moisturization benefits due to its ability to bind and hold water to the scalp and hair, preventing brittleness and breakage.

### In Vitro Study of Growth of Micro-Patterned 3D Hair Follicle Organoids Using Human Hair Follicle Cells

### 1. Cell Culture

Human Primary Dermal Papilla Cells (HFDPC) were collected from a 51-year-old male and provided by Kyungpook National University. Human Primary Epidermal Keratinocytes, neonatal foreskin (HEKn) cells were purchased from ATCC. HFDPC cells were seeded in a T-75 flask at 5×105 cells/dish and then cultured with Dulbecco's Modified solution containing 1% penicillin-streptomycin, 4 ng/mL Human basic fibroblast growth factor (hBFGF), and 10% Fetal bovine serum (FBS). HEKn cells were seeded in a T-75 flask coated with iMatrix at 2×105 cells/dish, incubated with Dermal Cell Basal Medium containing Keratinocyte Growth Kit supplement, and then cultured in an incubator (TC230, Thermo) containing 5% carbon dioxide at 37° C.

Cells were subcultured every 2 days, and the passage number of cells did not exceed 4.

### 2. Hair Follicle Organoid Growth Test

Hair follicle organoid is a 3D biological hair-like tissue that is composed of dermal papilla cell aggregates, which are the core tissues responsible for hair growth, surrounded by keratinocytes, mimicking natural hair growth in a laboratory setting. The hair follicle organoid growth test aims to mass-produces hair follicle organoids by proliferating dermal papilla cells isolated from adult hair tissue in vitro, mixing them with keratinocytes, and then co-culturing them on a specially designed particle pattern substrate (PAMCELL R300, 150 spheroids/well).

The experiment enables the assessment of the sample's impact on hair growth by monitoring changes in the diameter of keratinocyte pillars within the developed hair follicle organoid.

In this test, to differentiate and validate the growth of HFDPC and HEKn cells, HFDPC cells were stained with Cell tracker CM-Dil™ and HEKn cells were stained with Cell tracker Green CMFDA™. Stained HFDPC cells underwent filtration using a 15 μm strainer before being combined with DMEM medium to yield a density of 5×104 cells/well. Similarly, HEKn cells were adjusted to a density of 3×104 cells/well. Subsequently, 300 μL of the cell mixture was dispensed onto PAMCELL™ plates (96 well) to achieve a density of 8×104 cells/well, followed by a 24-hour incubation period in a CO2 incubator set at 37° C. After 24 hours of incubation, the medium was replaced with William's E medium containing 2 mM L-glutamin (100×), 10 ng/mL hydrocortisone, 10 μg/mL insulin, and 1% penicillin streptomycin. The cells were treated with Experimental Example 1 and Experimental Example 2 at concentrations of 0.5%, and 1%, respectively. The positive control group received treatment with minoxidil at a concentration of 1 μM.

Observations of hair follicle organoids were conducted over a 5-day period, with daily medium changes and sample processing. Results obtained on the 3rd day were utilized to evaluate the hair follicle organoid growth status and the sample's efficacy.

Hair follicle organoids pre- and post-treatment were imaged using a High-Content Screening (HCS, Cell3iMager duos2, SCREEN) device and then analyzed using the Cell 3 iMager Duos Software program (SCREEN). The untreated group, positive control group, and sample treatment group each consisted of 2 wells. The experimental results were calculated using Equation 1 and expressed as the hair follicle organoid growth rate on day 3 compared to the untreated group.

The results are presented as the average±standard deviation of the experiment.

$$Hair follicle organoid growth rate ( \% ) = \frac{treatment group Day 3 - treatment group Day 1}{untreated group Day 3 - untreated group Day 1} \times 100 \qquad [ Equation 1 ]$$

Untreated group: Group treated with culture medium only
Day 0: cell seeding
Day 1: Sample treatment by concentration
Day 2: Hair follicle organoid diameter on day 1 of sample treatment
Day 3: Hair follicle organoid diameter on day 2 of sample treatment

### 3. Data Analysis

1) Approximately 80% of hair follicle organoids generated in each well used in the test were used in the test were analyzed, and the results are expressed as mean #standard deviation.

2) The number of hair follicle organoids analyzed for each treatment group is shown below in Table 1.

TABLE 5

| | Numbers of hair follicle organoid (n) | |
|---|---|---|
| Untreated group | (n = 216) | |
| Experimental | 0.5% | (n = 215) |
| Example 1 | 1% | (n = 227) |
| Experimental | 0.5% | (n = 220) |
| Example 2 | 1% | (n = 225) |
| Minoxidil | 1 μM | (n = 234) |

3) Images were taken using the HCS device and the growth of hair follicle organoids was analyzed for 5 days using the Cell 3 iMager Duos Software program (SCREEN).

4) The test result measurements were initially analyzed using the Linear Mixed Model (LMM), which accounts for the random effects of each well. However, due to the lack of normality, the data were re-analyzed using the Generalized Linear Mixed Model (GLMM), which is suitable for non-normal data. Results showing significant differences between groups in the GLMM were subjected to Bonferroni correction (p<0.05, p<0.01, p<0.001).

### Results

### Hair Follicle Organoid Growth Rate

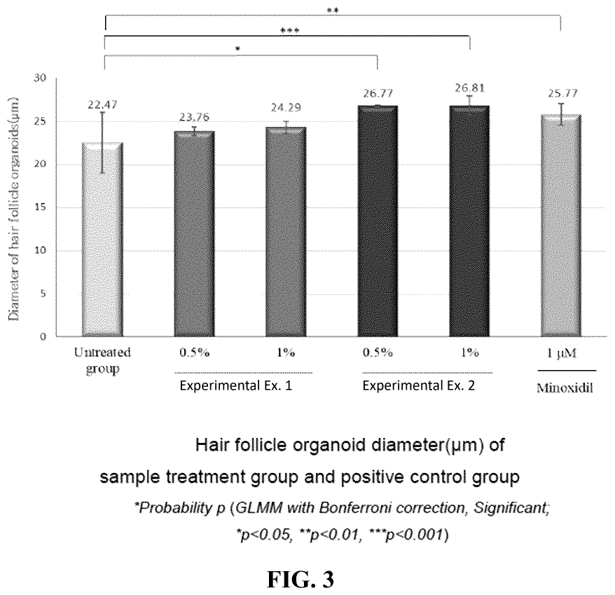

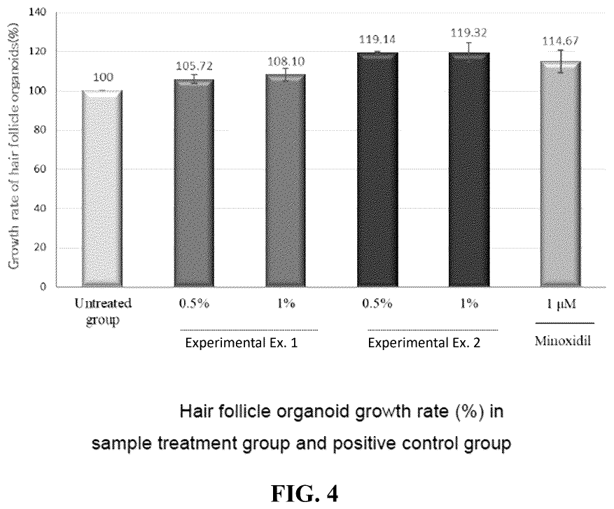

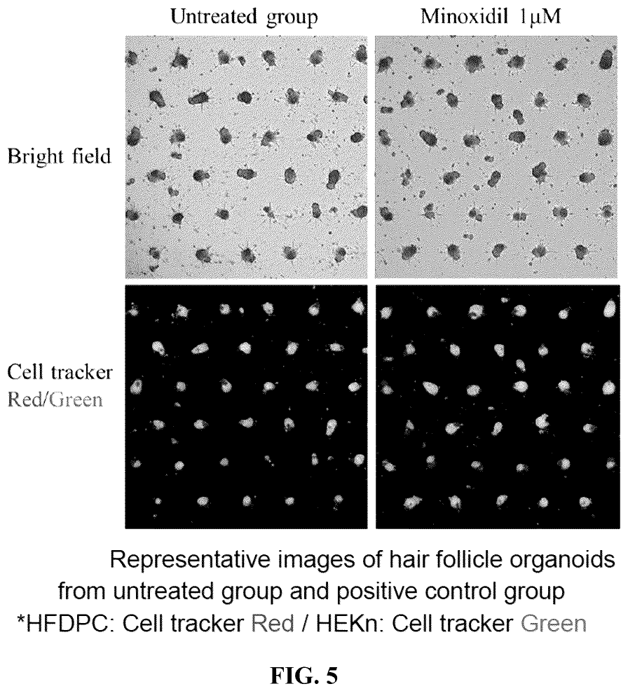

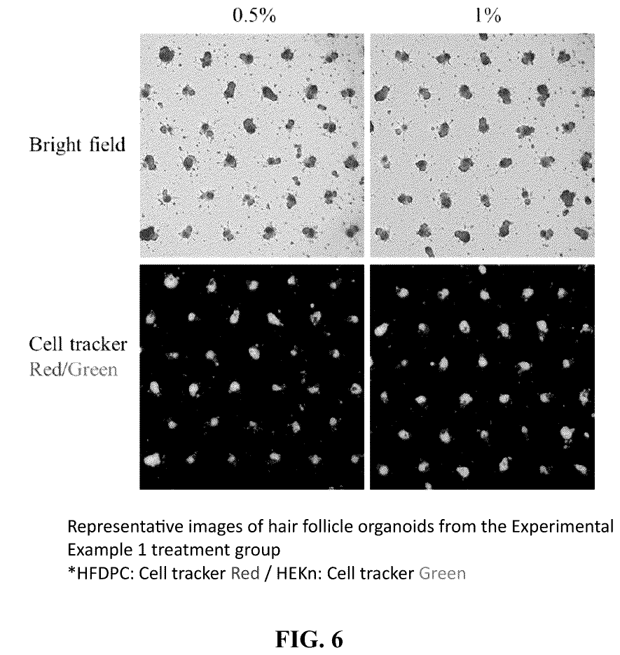

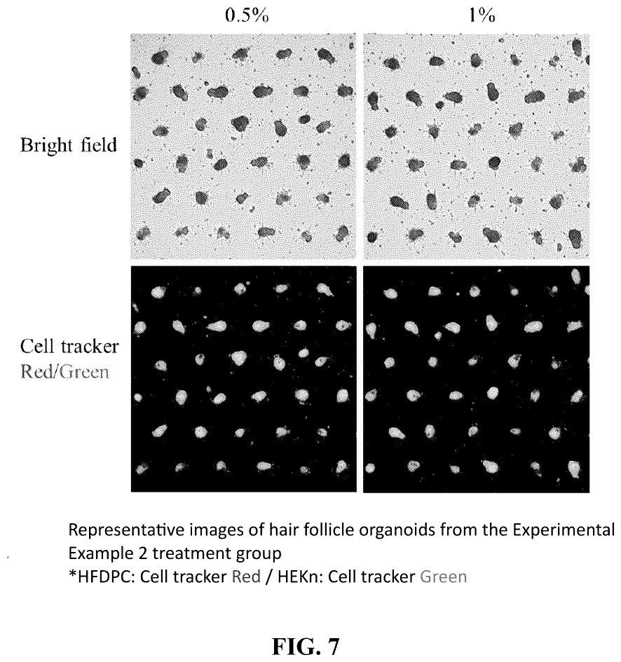

Hair follicle organoids cultured for 24 hours were treated with Experimental Example 1 and Experimental Example 2 at 0.5% and 1% concentrations, and minoxidil, a positive control, was treated at 1 μM concentration. The growth of hair follicle organoids was observed for 5 days, and the results from the 3rd day were used considering the growth status of hair follicle organoids and the effect of the sample. The diameter and growth rate of hair follicle organoids are shown in Tables 6 to 8, and the results are graphed as shown in FIGS. 3-4. Representative images are shown in FIGS. 5-7.

As a result of the test, the Experimental Example 1 treatment group showed a growth rate of hair follicle organoids of 105.72% at 0.5% concentration and 108.10% at 1% concentration compared to the untreated group.

The Experimental Example 2 treatment group showed a growth rate of hair follicle organoids of 119.14% at 0.5% concentration and 119.32% at 1% concentration compared to the untreated group.

Minoxidil, used as a positive control group, showed a hair follicle organoid growth rate of 114.67% at a concentration of 1 μM compared to the untreated group.

TABLE 6
Hair follicle organoid diameter and growth rate of Experimental
Example 1 (estimated mean ± standard deviation)

| | Untreated | Experimental Example 1 | |
|---|---|---|---|
| | group^{a} | 0.5% | 1% |
| hair follicle organoid | 22.47 ± 3.54 | 23.76 ± 0.50 | 24.29 ± 0.72 |
| diameter(μm) | | | |
| p-value^{b} | — | 1.030 | 0.394 |
| Hair follicle organoid | 100.00 ± 0.00 | 105.72 ± 2.23 | 108.10 ± 3.21 |
| growth rate (%) | | | |

^{a}Untreated group: cells treated with culture medium only
^{b}Probability p (GLMM with Bonferroni correction)

TABLE 7
Hair follicle organoid diameter and growth rate of Experimental
Example 2 (estimated mean ± standard deviation)

| | Untreated | Experimental Example 2 | |
|---|---|---|---|
| | group^{a} | 0.5% | 1% |
| hair follicle organoid | 22.47 ± 3.54 | 26.77 ± 0.14 | 26.81 ± 1.12 |
| diameter(μm) | | | |
| p-value^{b} | — | 0.033* | <0.001*** |
| Hair follicle organoid | 100.00 ± 0.00 | 119.14 ± 0.63 | 119.32 ± 4.97 |
| growth rate (%) | | | |

^{a}Untreated group: cells treated with culture medium only
^{b}Probability p (GLMM with Bonferroni correction, Significant; *p < 0.05, ***p < 0.001)

TABLE 8
Hair follicle organoid diameter and growth rate of Minoxidil
(positive control) (estimated mean ± standard deviation)

| | | Minoxidil |
|---|---|---|
| | Untreated group^{a} | 1 μM |
| hair follicle organoid | 22.47 ± 3.54 | 25.77 ± 1.28 |
| diameter(μm) | | |
| p-value^{b} | — | 0.009** |
| Hair follicle organoid | 100.00 ± 0.00 | 114.67 ± 5.69 |
| growth rate (%) | | |

^{a}Untreated group: cells treated with culture medium only
^{b}Probability p (GLMM with Bonferroni correction, Significant; **p < 0.01)

The above data from Tables 6-8 are also presented in graph format in FIGS. 3-4, and results images of the hair follicle organoid diameter and growth are presented in FIGS. 5-7.

In summary of the in vitro experiments pertaining to the effect of the Experimental Examples 1 and 2 of the present disclosure with respect to hair follicle organoid diameter and growth, it can be seen that Experimental Example 1 treatment group showed a growth rate of hair follicle organoids of 105.72% at 0.5% concentration and 108.10% at 1% concentration compared to the untreated group. The Experimental Example 2 treatment group showed a growth rate of hair follicle organoids of 119.14% at 0.5% concentration and 119.32% at 1% concentration compared to the untreated group.

Minoxidil, used as a positive control group, showed a hair follicle organoid growth rate of 114.67% at a concentration of 1 μM compared to the untreated group.

Therefore, as a result of testing the hair growth promotion effect of the test materials using mass-produced 3D co-cultured hair follicle hair tissue, it was confirmed that both Examples 1 and 2 were effective concentrations of 0.5% and 1% to provide greater hair follicle organoid diameter and growth rate compared to the samples of the untreated group.

### Applications

After the postbiotic fermentation composition is obtained using Lactobacillus plantarum and/or Lactobacillus casei fermentation, it may be used in a variety of different applications. As one non-limiting example, the postbiotic ferment composition obtained after fermentation of eight different extracts and/or raw materials may be provided in broth form and combined with butylene glycol and 1,2-hexanediol to obtain a topical composition. The topical composition may include ˜92 wt % of the fermentation product, ˜5% of butylene glycol, and ˜3% of 1,2-hexanediol. The topical composition may have a pH value of 4.0-7.0, more specifically 5.22. The topical composition was shown to be safe for use on the root, scalp, and hair, measuring less than 100 CFU/mL of bacteria, and less than 10 CFU/mL of yeast/mold. No pathogens were detected from the microbes of the topical composition. Thus, the topical composition measured to be safe for clinical trials, measuring less than 10 ppm of heavy metals, including lead, arsenic, antimony, cadmium, mercury, and nickel.

In another example, the postbiotic ferment composition including fermentation of eight different ingredients may be provided in powder form by combining with maltodextrin. The powder composition may include ˜80% of the fermentation product and ˜20% of maltodextrin. The powder composition also showed measurements to be safe for human use, the powder composition having less than 100 CFU/g of bacteria, and less than 10 CFU/g of yeast/mold. No pathogens were detected from the microbes of the powder composition. Thus, the powder composition measured to be safe for clinical trials, measuring less than 10 ppm of heavy metals.

In yet another example, the postbiotic ferment composition may be used to form ingestible dietary supplements for internal consumption, such as, but not limited to, gummies, capsules, elixirs, or beverages. The nutraceutical made from the postbiotic ferment composition of the present disclosure could be used to support root, scalp, hair, and other parts of the skin via the gut microbiome (i.e., the gut-skin axis) by reinforcing the gut barrier, bolstering immune functioning, antioxidant protection while soothing inflammation.

While the focus of the present disclosure is on treatment of root, scalp and hair care, aspects of the present disclosure may be adapted to be used for applications of other parts of the body to improve the health and wellness of the facial and body skin. The postbiotic ferment composition of the present disclosure is capable of supporting microbiome balance and health, reinforcing the barrier, antioxidant protection, improving skin tone and texture, and reducing irritations that affect skin in general, and not just for the scalp and hair.

Except where a contrary intent is expressly stated, the terms “comprises,” “comprising,” “containing,” and “having” and the like mean “includes,” “including,” and the like, and are generally interpreted to be open ended transition terms. The recitation of components, structures, steps, or the like specifically listed following an open-ended transition term in no way limit such claim to the components, structures, steps, or the like specifically listed. The terms “consisting of’ or “consists of’ are closed transition terms.

Except where a contrary intent is expressly stated, terms are used in their singular form for clarity and are intended to include their plural form.

Occurrences of the phrase “in one embodiment,” or “in one aspect,” herein do not necessarily all refer to the same embodiment or aspect.

While this invention has been described as having designs illustrated by embodiments and examples, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

1. A postbiotic ferment composition for treatment of root, scalp and/or hair, wherein the postbiotic ferment composition is prepared by fermenting a combined product comprising an extract obtained from a plurality of raw materials, Pisum sativum, and Oryza sativa,
wherein the plurality of raw materials comprises Panax ginseng root, Ganoderma lucidum, Centella asiatica, coffee arabica seed, Scutellaria baicalensis root, and Humulus lupulus, and
wherein the combined product is fermented using one or more bacterial species of Lactobacillus.

2. The composition of claim 1, wherein the one or more bacterial species of Lactobacillus is selected from the group consisting of Lactobacillus plantarum and Lactobacillus casei.

3. The composition of claim 2, wherein the one or more bacterial species of Lactobacillus is both Lactobacillus plantarum and Lactobacillus casei.

4. The composition of claim 3, wherein the extract of the plurality of raw materials is obtained from 2-4 wt % Centella asiatica, 2-4 wt % Humulus lupulus, 2-5 wt % Scutellaria baicalensis root, 1-5 wt % Panax ginseng root, 1-4 wt % Ganoderma lucidum, and 0.25-2 wt % coffee arabica seed, and
wherein the extract is combined with 0.25-1 wt % Pisum sativum and 0.25-1 wt % Oryza sativa.

5. The composition of claim 3, wherein the extract of the plurality of raw materials is obtained from 2.5 wt % Centella asiatica, 2.5 wt % Humulus lupulus, 2.5 wt % Scutellaria baicalensis root, 1 wt % Panax ginseng root, 1 wt % Ganoderma lucidum, and 0.5 wt % coffee arabica seed, and
wherein the extract is combined with 0.25 wt % Pisum sativum, and 0.25 wt % Oryza sativa.

6. A method of preparing a postbiotic ferment composition for treatment of root, scalp and/or hair, the method comprising:
obtaining an extract from a plurality of raw materials, wherein the plurality of raw materials comprises Panax ginseng root, Ganoderma lucidum, Centella asiatica, Humulus lupulus, coffee arabica seed, and Scutellaria baicalensis root;
combining the obtained extract with Pisum sativum and Oryza sativa to obtain a combined product; and
inoculating the combined product with one or more bacterial species of Lactobacillus to induce fermentation and obtain the postbiotic ferment composition for treatment of root, scalp, and/or hair.

7. The method of claim 6, wherein the one or more bacterial species of Lactobacillus is selected from the group consisting of Lactobacillus plantarum and Lactobacillus casei.

8. The method of claim 7, wherein the one or more bacterial species of Lactobacillus is both Lactobacillus plantarum and Lactobacillus casei.

9. The method of claim 8, wherein the extract of the plurality of raw materials is obtained from 2-4 wt % Centella asiatica, 2-4 wt % Humulus lupulus, 2-5 wt % Scutellaria baicalensis root, 1-5 wt % Panax ginseng root, 1-4 wt % Ganoderma lucidum, and 0.25-2 wt % coffee arabica seed, and
wherein the extract is combined with 0.25-1 wt % Pisum sativum and 0.25-1 wt % Oryza sativa.

10. The method of claim 8, wherein the extract of the plurality of raw materials is obtained from 2.5 wt % Centella asiatica, 2.5 wt % Humulus lupulus, 2.5 wt % Scutellaria baicalensis root, 1 wt % Panax ginseng root, 1 wt % Ganoderma lucidum, and 0.5 wt % coffee arabica seed, and
wherein the extract is combined with 0.25 wt % Pisum sativum, and 0.25 wt % Oryza sativa.

11. The method of claim 10, wherein the combined product is inoculated with 0.5% to 1% w/w Lactobacillus plantarum and 0.5% to 1% w/w Lactobacillus casei.

12. A method of treating root, scalp and/or hair comprising: topically applying the composition of claim 1.

13. A method of treating root, scalp and/or hair comprising: ingesting a nutraceutical comprising the composition of claim 1.
